(19) United States
(12) Patent Application Publication
(10) Pub. No.: US 2010/0324617 A1
(43) Pub. Date: Dec. 23, 2010

(54) ADAPTER FOR ELECTROSTIMULATION LEAD AND METHOD FOR REDUCING EXTRACARDIAC STIMULATION

(76) Inventor: James J. Ong, Woodland Hills, CA (US)

Correspondence Address: ITALIA IP, 3500 WEST OLIVE AVE., SUITE 300, BURBANK, CA, 91505, US

(21) Appl. No.: 12/486,162

(22) Filed: Jun. 17, 2009

(51) Int. Cl. A61N 1/362; H01R 11/00

(52) U.S. Cl. 607/9; 439/502

(57) ABSTRACT

A method of cardiac electrostimulation of a patient with a cardiac electrostimulation device includes implanting a first electrode in the left ventricle of the patient. A second electrode is implanted in the heart of the patient. In some embodiments, the first electrode and second electrode are electrically connected in parallel and share a common anode. The method reduces the likelihood of phrenic stimulation in the patient. Adapters and electrostimulation devices are also disclosed.

### BACKGROUND OF INVENTION

This application relates to the field of pacemakers (PM) and implantable defibrillators (ICD) and in particular devices and methods of delivering Cardiac Resynchronization Therapy (CRT) in patients who may experience intractable phrenic nerve stimulation during CRT.

### SUMMARY

Permanent cardiac electrostimulation, such as permanent PMs and ICDS, are bioelectrical devices designed to monitor and maintain heart rates of patients with either too slow of a heart rate or too fast of a heart rate. These devices are comprised of two basic components, namely a pulse generator contained within a casing, which is subcutaneously implanted, and leads which extend from the pulse generator through the veins and into the heart chamber. The leads function as the pathway for the electrostimulation device to deliver electrical impulses to the heart muscle in order to maintain a normal heart rate.

The leads are generally defined as either unipolar or bipolar. A unipolar lead is a lead having one electrode, the cathode, at its distal tip. To complete the electrical circuit, the other electrode, the anode, is at the pulse generator located on the chest. Bipolar leads, on the other hand, have both the cathode and anode at or near the end of the lead within the heart chamber. The distal electrode of the pair is the cathode, the proximal the anode.

Typical electrostimulation systems consist of two leads, one in the Right Atrium (RA) and one in the Right Ventricle (RV). This is referred to as a “dual chamber” PM or ICD. It maintains synchrony between the atrium and the ventricle. However, synchrony between the right and left ventricle is not maintained, and, in fact, is frequently disturbed, when pacing occurs through the right ventricle only. Creation of such dysynchronization between the ventricles frequently leads to heart failure.

CRT is a method of delivering pacing in both the right and left ventricles, and thus is commonly referred to as “biventricular pacing.” CRT may be particularly useful in patients with failing hearts, in which the electrical and mechanical function have lost the usual “synchrony” that is seen in healthy heart. Restoration of this synchrony is the goal in CRT.

CRT is achieved by, in addition to the standard RA and RV leads in a dual chamber PM or ICD, implanting an additional electrode in the left ventricle (LV) either epicardially via a thoracotomy or transvenously via the coronary sinus venous system. Resynchronization is thus achieved by pacing, simultaneously, or with a preset small time difference, both the RV and LV.

One limitation to the successful implantation of an LV lead, and thus, the successful delivery of CRT, is the presence of phrenic nerve stimulation. The phrenic nerve is a nerve that controls the movement of the diaphragm and is responsible for important activities such as breathing, as well as annoyances such as hiccups. The nerve runs along side of the heart and is often in close proximity to where the LV lead is implanted. As a result, stimulating the heart through the LV lead can lead to intractable hiccuping if the lead is too close to the phrenic nerve.

As previously mentioned, the LV lead may be implanted transvenously through the coronary sinus venous system. Since the venous anatomy for each patient is generally fixed, a surgeon usually does not have many options where to implant the LV lead. Even if there is more than one vein, other veins are also frequently associated with phrenic nerve stimulation. Successful insertion is also limited by the size and contour of the vein. Additionally, not all veins are suitable for LV pacing. Even with epicardial implantation of an LV electrode, phrenic nerve stimulation may be inevitable. Thus, in the case of phrenic nerve stimulation, it frequently means CRT failure. This application discloses devices and methods for reducing the amount of and/or instances of phrenic nerve stimulation whilst delivering CRT.

In particular, this application discloses an adapter for coupling two or more electrodes to an electrostimulation device, the adapter comprising: a yoke portion; a connector portion extending distally from the yoke portion, the connector portion including a bipolar lead connector having a ring and a tip, wherein the connector portion is configured for reception by an electrostimulation device; a first receptacle configured to accept an electrode and connected to the yoke portion, the first receptacle including a ring and a tip, wherein the ring is in electrical communication with the connector portion ring, and wherein the tip is in electrical communication with the connector portion tip; and a second receptacle configured to accept an electrode and connected to the yoke portion, the second receptacle having a ring and a tip, wherein one of the tip and the ring is in electrical communication with the connector portion tip, and wherein the other of the tip and the ring is electrically isolated.

This application further discloses a method of cardiac electrostimulation of a patient with a cardiac electrostimulation device, the method comprising: implanting a first electrode in the patient; and implanting a second electrode in the left ventricle of the patient, wherein the first electrode and second electrode are electrically connected in parallel, and wherein the first electrode and second electrode share a common anode.

This application also discloses a cardiac electrostimulation device comprising: a cardiac electrostimulator; a first receptacle for receiving a first electrode, the first receptacle in electrical communication with the electrostimulator, the first receptacle including a ring portion and a tip portion; a second receptacle for receiving a second electrode, the second receptacle in electrical communication with the electrostimulator, the second receptacle including a ring portion and a tip portion; a third receptacle for receiving a third electrode for implantation within the left ventricle of a patient, the third receptacle in electrical communication with the electrostimulator, the third receptacle including a tip portion; and a fourth receptacle for receiving a fourth electrode, the fourth receptacle in electrical communication with the electrostimulator, the fourth receptacle including a ring portion and a tip portion.

This application further discloses a cardiac electrostimulation device comprising: a cardiac electrostimulator; a first receptacle for receiving a first electrode, the first receptacle in electrical communication with the electrostimulator, the first receptacle including a ring portion and a tip portion; a second receptacle for receiving a second electrode, the second receptacle in electrical communication with the electrostimulator, the second receptacle including a ring portion and a tip portion; a third receptacle for receiving a third electrode for implantation within the left ventricle of a patient, the third receptacle in electrical communication with the electrostimulator, the third receptacle including a ring portion; and a fourth receptacle for receiving a fourth electrode, the fourth receptacle in electrical communication with the electrostimulator, the fourth receptacle including a ring portion and a tip portion.

This application also discloses an adapter for coupling an electrode to an electrostimulation device, the adapter comprising: a connector portion having a unipolar lead with a tip, wherein the connector portion is configured for reception by an electrical stimulation device; and a receptacle configured to accept an electrode and connected to the connector portion, the receptacle having a ring and a tip, wherein the ring is in electrical communication with the connector portion tip, and wherein the receptacle tip is in electrical isolation.

Moreover, this application discloses an adapter for coupling an electrode to an electrostimulation device, the adapter comprising: a connector portion including a bipolar lead connector having a ring and a tip, wherein the connector portion is configured for reception by an electrostimulation device, wherein the ring is configured for electrical communication with an anode of the electrostimulation device, and wherein the tip is configured for electrical communication with a cathode of the electrostimulation device; and a receptacle configured to accept an electrode and in electrical communication with the connector portion, the receptacle including a ring and a tip, wherein the ring is in electrical communication with the connector portion tip, and wherein the tip is in electrical communication with the connector portion ring.

### BRIEF DESCRIPTION OF THE DRAWINGS

The drawings, when considered in connection with the following description, are presented for the purpose of facilitating an understanding of the subject matter sought to be protected.

FIG. 1 is a plan view of an adapter for coupling two or more electrodes to an electrostimulation device;

FIG. 2A is a schematic view of a receptacle of FIG. 1;

FIG. 2B is a schematic view of an alternative receptacle of FIG. 1;

FIG. 3A is a schematic view of an electrostimulation device;

FIG. 3B is a schematic view of an alternative electrostimulation device;

FIG. 4A is a plan view of an adapter for coupling an electrode to an electrostimulation device;

FIG. 4B is a schematic view of a receptacle of FIG. 3A; and

FIG. 5 is a schematic view of an adapter for coupling an electrode to an electrostimulation device.

### DETAILED DESCRIPTION

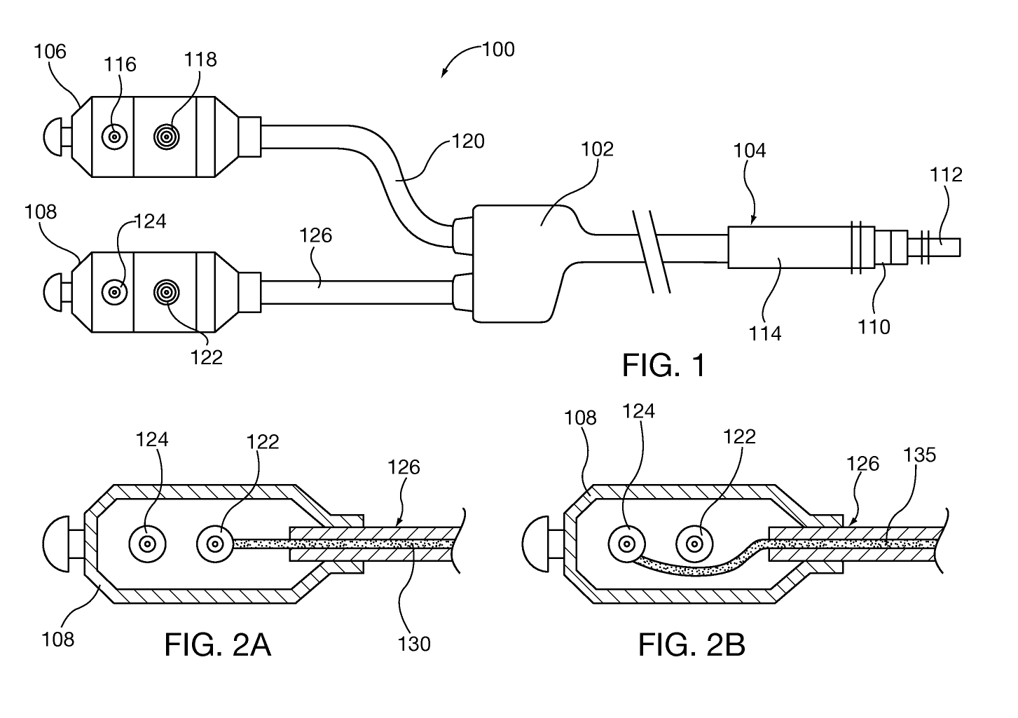

Referring now to FIG. 1, an illustrative adapter 100 for electrically coupling two or more electrodes to an electrostimulation device, such as a pacemaker or implantable defibrillator, is shown. The adapter 100 generally includes a yoke portion 102, a connector portion 104, a first receptacle 106 and a second receptacle 108. The yoke portion 102 provides electrical communication between the connector portion 104 and the first and second receptacles 106, 108. The connector portion 104 includes a bipolar lead connector 114 having a ring 110 and a tip 112. The bipolar lead connector 114 may be any suitable bipolar, or multipolar, lead connector, including, but not limited to, IS1, IS4, LV1, 6 mm, or any other suitable bipolar, or multipolar, lead connector. The connector portion 104 extends distally from the yoke portion 102 and is configured to be received by a receptacle of a electrostimulation device whereby electrical signals are transmitted from the electrostimulation device to the connector portion 104, wherein the ring 110 electrically communicates with the anode portion of the electrostimulation device and wherein the tip 112 electrically communicates with the cathode portion of the electrostimulation device

The first receptacle 106 is configured to accept a bipolar, or multipolar, electrode that provides electrical stimulation to the target tissue, including, but not limited to, heart tissue. Illustrative electrodes include, without limitation, IS1, IS4, LV1, 6 mm, etc. The first receptacle 106 includes a ring 116 and a tip 118. A lumen 120 extends between the first receptacle 106 and yoke portion 102. The lumen 120 provides electrical communication between the first receptacle 106 and the yoke portion 102. The lumen 120 includes an electrically conductive core (not shown) that provides, via the yoke, electrical communication between the tip 118 and the connector portion tip 112. Additionally, the lumen 120 includes an electrically conductive outer coil (not shown) that provides, via the yoke, electrical communication between the ring 116 and the connector portion ring 110. The core and outer coil of the lumen 120 are insulated from one another. The tip 118 is operative to receive a corresponding tip of an electrode and the ring 116 is operative to receive a corresponding ring of an electrode. Thus, the electrode tip may be in electrical communication with the connector portion tip 112, such that the electrode tip may be in electrical communication with the electrostimulation device cathode, and the electrode ring may be in electrical communication with the connector portion ring 110, such that the electrode ring may be in electrical communication with the electrostimulation device anode. An electrode coupled to the first receptacle 106 may be implanted in the right ventricle (RV), left ventricle (LV), superior vena cava (SVC), a second cardiac vein, coronary sinus (CS), or the great cardiac vein. It will, however, be appreciated that an electrode extending from the receptacle 106 may be a “fourth electrode” and may be implanted in any suitable location and such suitable locations are not limited to the forgoing examples. The term “fourth electrode” will be appreciated to convey that an electrode extending from the receptacle 106 (i.e. the “fourth electrode”) may be used in conjunction with a left ventricle electrode (as will be described below) as well as conventional right atrium and right ventricle electrodes. Nevertheless, it will be appreciated that the term “fourth electrode” is illustrative only and not limiting in any way and in no way limits the present disclosure to embodiments employing just four electrodes. For example, an electrode extending from the first receptacle 106 may be used in conjunction with a left ventricle electrode (as will be described below) and a conventional right ventricle electrode. Alternatively, any suitable number of conventional electrodes may be employed with an electrode extending from the first receptacle 106 and a left ventricle electrode (as will be described below).

The second receptacle 108 may be configured to accept either a bipolar, multipolar, or unipolar electrode for providing electrical stimulation to the target tissue, such as heart tissue. Suitable electrodes, include, without limitation, IS1, IS4, LV1, DF1, 6 mm, etc. The second receptacle 108 may include a ring 124, configured to receive an electrode ring, and a tip 122, configured to receive an electrode tip. A lumen 126 provides electrical communication between the second receptacle 108 and the yoke portion 102.

Either the tip 122 or ring 124 is in electrical communication with the connector portion tip 112 and the other one of the pair (i.e., the tip 122 or ring 124) is electrically isolated. In some embodiments, the tip 122 or ring 124 that is in electrical communication with the connector portion tip 112 is also electrically connected in parallel with the first receptacle tip 118. Additionally, in one embodiment, the first receptacle ring 116 acts as a common anode for an an electrode in communication with the first receptacle 106 and an electrode in communication with the second receptacle 108. Advantageously, by coupling the LV electrode to the second receptacle 108, extra cardiac stimulation, such as phrenic nerve stimulation, may be diminished or eliminated.

In one embodiment, the tip 122 is in electrical communication with the connector portion tip 112 and the ring 124 is electrically isolated. In the illustrative embodiment of FIG. 2A, the lumen 126 does not include a conductive outer coil and the core 130 of the lumen 126 provides electrical communication between the tip 122 and the connector portion tip 112 via the yoke 102. In an alternative embodiment, the conductive outer coil of the lumen 126 is terminated in the second receptacle 108 such that the conductive outer coil and ring 124 are not in electrical communication. Electrical communication between the tip 122 and the connector portion tip 112 is provided by the conductive core 130 of the lumen 126 and the yoke 102. It will be appreciated that the tip 122 and connector portion tip 112 may be electrically connected by any suitable means and the present disclosure is in no way limited to the illustrative schematic.

Additionally, in one embodiment, the second receptacle tip 122 and first receptacle tip 118 are electrically connected in parallel via the yoke 102. Further, in one embodiment, the first receptacle ring 116 acts as a common anode for an electrode extending from the second receptacle 108 and an electrode extending from the first receptacle 106. As mentioned, an electrode extending from the second receptacle 108 is implanted in the LV.

Referring again to FIG. 1, in another embodiment, the ring 124 is in electrical communication with the connector portion tip 112 and the second receptacle tip 122 is electrically isolated. In one embodiment, as shown in FIG. 2B, the lumen 126 does not include a conductive core and an outer coil. Rather, the lumen 126 includes a conductive wire 135, which provides electrical communication between the ring 124 and the connector portion tip 112 via the yoke 102. In an alternative embodiment, the conductive outer coil of the lumen 126 is terminated in the second receptacle 108 such that the conductive outer coil and ring 124 are not in electrical communication. Electrical communication between the ring 124 and the connector portion tip 112 is provided by the conductive core of the lumen 126 and via the yoke 102. It will be appreciated that the ring 124 and connector portion tip 112 may be electrically connected by any suitable means and the present disclosure is in no way limited to the illustrative schematic.

Additionally, in one embodiment, the second receptacle ring 124 and first receptacle tip 118 are electrically connected in parallel via the yoke 102, whereby the second receptacle ring 124 and first receptacle tip 118 are in electrical communication with a cathode of the electrostimulation device. Further, in one embodiment, the first receptacle ring 116 acts as a common anode for an electrode extending from the second receptacle 108 and an electrode extending from the first receptacle 106. As mentioned, an electrode extending from the second receptacle 108 is implanted in the LV.

Optionally, one or more adjustable resistors (not shown) may be in electrical communication with the connector portion and one or both of the receptacles 106, 108. Advantageously, the resistance may be adjusted by an operator implanting leads extending from the receptacle(s). By adjusting the resistance via the adjustable resistors, the current density at each lead may be adjusted such that extra cardiac stimulation may be minimized. Also, it will be appreciated that adjustable resistors for controlling current densities for various electrodes may be employed with any suitable connector for coupling one or more electrodes to an electrostimulation device and the use of adjustable resistors for the purpose of controlling current densities of one or more electrodes is not limited to the forgoing connectors and the use of adjustable resistors with any suitable connector is expressly contemplated by the present disclosure.

A method for providing cardiac electrostimulation to a patient whilst reducing, or eliminating, the likelihood of phrenic nerve stimulation, or other extracardiac stimulation, is also provided. The method includes implanting a first electrode in, or near, the heart of a patient. The first electrode may be bipolar or multipolar. The first electrode may include a ring portion, for electrical communication with with an anode of an electrostimulation device, and a tip portion, for electrical communication with a cathode of an electrostimulation device. The first electrode may be implanted in any suitable location, including, but not limited to, the right ventricle (RV), left ventricle (LV), superior vena cava (SVC), a second cardiac vein, coronary sinus (CS), or great cardiac vein. A second electrode is implanted in the left ventricle of the patient. The second electrode may be unipolar, bipolar or multipolar. The second electrode may include one or both of a ring portion and a tip portion. In one embodiment, the tip portion of the first electrode is electrically connected in parallel with a tip portion of the second electrode and the ring of the second electrode is electrically isolated. The ring of the first electrode may serve as a common anode for the first and second electrodes. In an alternative embodiment, the tip portion of the first electrode is electrically connected in parallel with a ring portion of the second electrode and the tip of the second electrode is electrically isolated. The ring of the first electrode may serve as a common anode for the first and second electrodes. The method may also include implanting an electrode in the right ventricle of the patient and/or implanting an electrode in the right atrium of the patient.

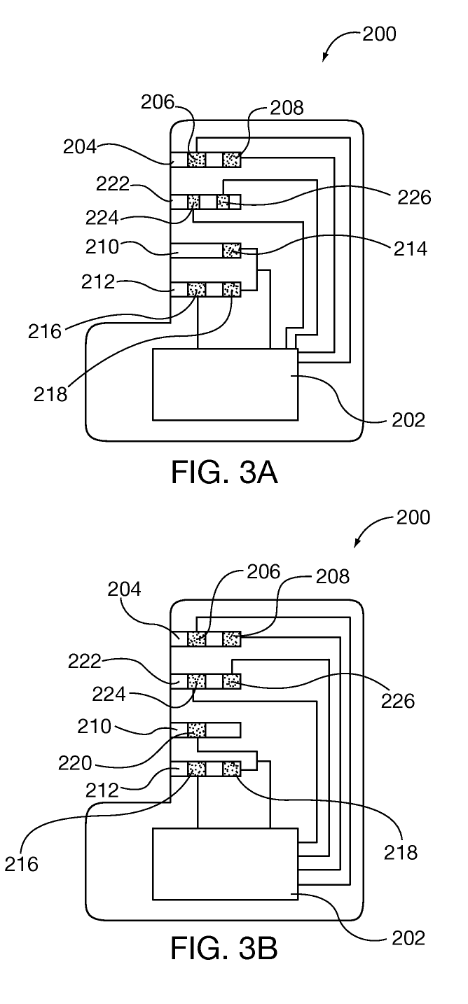

Referring now to FIG. 3A, a cardiac electrostimulation device 200 is shown. The device 200 includes a cardiac electrostimulator generally represented by block 202. The electrostimulator 202 may be an ICD, a PM, or any other suitable stimulation device. The electrostimulator 202 may include circuitry, computer(s), one or more batteries, or any other components for electrically stimulating the heart of a patient.

The device 200 may also include a first receptacle 204 having a ring portion 206 and a tip portion 208. The first receptacle 204 is in electrical communication with the electrostimulator 202, wherein the ring portion 206 is in electrical communication with an anode of the electrostimulator 202 and wherein the tip portion 208 is in electrical communication with the cathode of the electrostimulator 202. The first receptacle 204 may be configured to receive a first electrode and provide electrical communication between the electrostimulator 202 and the first electrode. Illustrative first electrodes, include, without limitation, IS1, IS4, LV1, 6 mm, etc. An electrode in electrical communication with the first receptacle 204 may be implanted in any suitable location within the heart of a patient, including, but not limited to, the right atrium, right ventricle, etc. It will be appreciated that the first receptacle is optional and a device not having such a first receptacle is still encompassed by the present disclosure. Additionally, it will be appreciated that the device may include any suitable number of receptacles configured as the illustrative first receptacle 204 and remain within the scope of the present disclosure.

The device 200 may also include a second receptacle 222 having a ring portion 224 and a tip portion 226. The second receptacle 222 is in electrical communication with the electrostimulator 202, wherein the ring portion 224 is in electrical communication with an anode of the electrostimulator 202 and wherein the tip portion 226 is in electrical communication with a cathode of the electrostimulator 202. The second receptacle 222 may be configured to receive a second electrode and provide electrical communication between the electrostimulator 202 and the second electrode. Illustrative second electrodes, include, without limitation, IS1, IS4, LV1, 6 mm, etc. An electrode in electrical communication with the second receptacle 222 may be implanted in any suitable location within the heart of a patient, including, but not limited to, the right atrium, right ventricle, etc. It will be appreciated that the second receptacle is optional and a device not having such a second receptacle is still encompassed by the present disclosure. Additionally, it will be appreciated that the device may include any suitable number of receptacles configured as the illustrative second receptacle 222 and remain within the scope of the present disclosure.

The device 200 also includes a third receptacle 210 and a fourth receptacle 212. The third receptacle 210 is in electrical communication with the electrostimulator 202. The third receptacle 210 includes a tip portion 214 wherein the tip portion 214 is in electrical communication with a cathode of the electrostimulator 202. The third receptacle 210 is configured to receive a third electrode for implantation within the left ventricle of a patient and provide electrical communication between the third electrode and electrostimulator 202. Illustrative third electrodes, include, without limitation, IS1, IS4, LV1, 6 mm, etc.

The fourth receptacle 212 includes a ring portion 216 and a tip portion 218. The fourth receptacle 212 is in electrical communication with the electrostimulator 202 wherein the ring portion 216 is in electrical communication with an anode of the electrostimulator 202 and wherein the tip portion 218 is in electrical communication with a cathode of the electrostimulator 202. Additionally, in one embodiment, the tip portion 218 is electrically connected in parallel with the third receptacle tip portion 214 such that the ring portion 216 of the fourth receptacle serves as a common anode for a third electrode coupled to the third receptacle 210 and a fourth electrode coupled to the fourth receptacle 212. The fourth receptacle 212 is configured to receive a fourth electrode for implantation within the heart of the patient and provide electrical communication between the fourth electrode and electrostimulator 202. Illustrative fourth electrodes, include, without limitation, IS1, IS4, LV1, 6 mm, etc. Moreover the fourth electrode may be implanted in any suitable location within the heart of the patient, including, but not limited to, the right ventricle (RV), left ventricle (LV), superior vena cava (SVC), a second cardiac vein, coronary sinus (CS), or the great cardiac vein.

With particular reference to FIG. 3B, an alternative device 200 is shown. The first and second receptacles 204, 222 may be configured as described with respect to FIG. 3A. The device 200 may be configured such that the third receptacle 210 includes a ring portion 220 and no tip portion. The third receptacle ring portion 220 is in electrical communication with a cathode of the electrostimulator 202. Additionally, in one embodiment, the third receptacle ring portion 220 may be electrically connected in parallel with the fourth receptacle tip portion 218. Also, in one embodiment, the ring portion 216 of the fourth receptacle 212 may serve as a common anode for respective electrodes in communication with the third receptacle 210 and fourth receptacle 212. An electrode in communication with the third receptacle 210 may be implanted within the left ventricle of a patient and an electrode in communication with the fourth receptacle 212 may be implanted in any suitable location within the heart of the patient, including, but not limited to, the right ventricle (RV), left ventricle (LV), superior vena cava (SVC), a second cardiac vein, coronary sinus (CS), or the great cardiac vein.

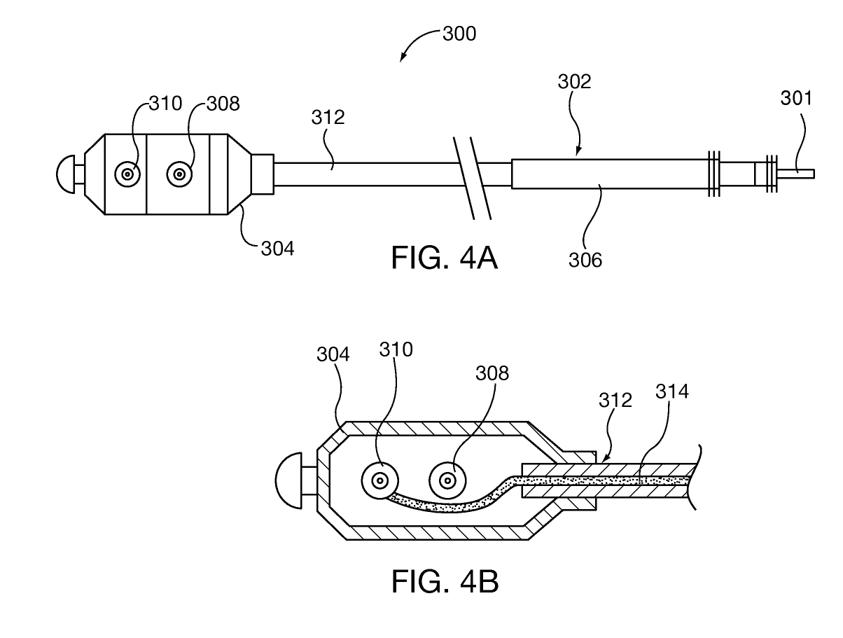

Referring now to FIGS. 4A-4B, another adapter 300 for coupling an electrode to an electrostimulation device is shown. The adapter 300 includes a connector portion 302 and a receptacle 304. The connector portion 302 includes a unipolar lead connector 306 and a tip 301 for electrically communicating with a cathode of an electrostimulation device. The connector portion 302 is configured to be received by a receptacle of a electrostimulation device whereby electrical signals are transmitted from the electrostimulation device to the connector portion 302 and ultimately through an electrode in electrical communication with the receptacle 304.

The receptacle 304 is configured to accept a bipolar, or multipolar, electrode that provides electrical stimulation to the target tissue, including, but not limited to, heart tissue. Illustrative electrodes, include, without limitation, IS1, IS4, LV1, 6 mm, etc. The receptacle 304 includes a tip 308 and a ring 310. The tip 308 is configured to receive an electrode tip. Also, the ring 310 is adapted to receive an electrode ring. A lumen 312 extends between the receptacle 304 and the connector portion 302. The lumen 312 includes an electrically conductive core 314 (see FIG. 4B) which provides electrical communication between the ring 310 and the connector portion tip 301. Therefore, the receptacle ring 310 is in electrical communication with a cathode of the electrostimulation device and the tip 308 is electrically isolated such that the ring of an electrode in communication with the receptacle 304 acts as the cathode for the electrode. Advantageously, the adapter 300 may be used where the tip of the electrode initially causes phrenic nerve stimulation; wherein, this adapter 300 reverses the polarity of the electrode thereby utilizing the electrode ring as the preferred pacing site. This may be useful when an electrostimulation device does not include “electronic repositioning” capability.

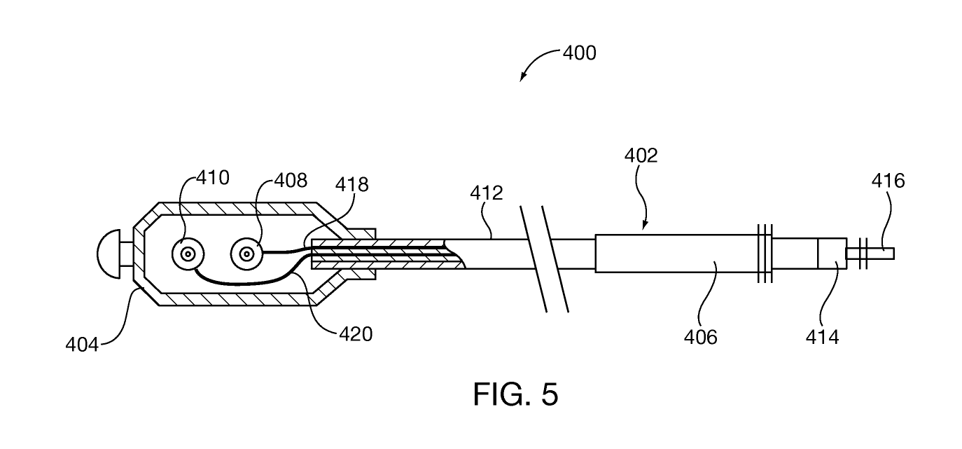

Referring now to FIG. 5, another adapter 400 for coupling an electrode to an electrostimulation device is shown. The adapter 400 includes a connector portion 402 and a receptacle 404. The connector portion 402 includes a bipolar lead connector 406 having a ring 414 and a tip 416. The lead connector 406 is configured to be received by an electrostimulation device such that the ring 414 is in electrical communication with an anode of the electrostimulation device and the tip 416 is in electrical communication with a cathode of the electrostimulation device, whereby electrical signals are transmitted from the electrostimulation device to the connector portion 402 and ultimately through an electrode in electrical communication with the receptacle 404.

The receptacle 404 is configured to accept a bipolar, or multipolar, electrode that provides electrical stimulation to the target tissue, including, but not limited to, heart tissue. Illustrative electrodes, include, without limitation, IS1, IS4, LV1, 6 mm, etc. The receptacle 404 includes a tip 408 and a ring 410. The tip 408 is configured to receive an electrode tip and the ring 410 is configured to receive an electrode ring. A lumen 412 extends between the receptacle 404 and the connector portion 402. The lumen 412 includes a first electrically conductive portion 420, such as a wire, which provides electrical communication between the receptacle ring 410 and the connector portion tip 416, and a second electrically conductive portion 418, such as a second wire which may be insulated from the first wire, which provides electrical communication between the receptacle tip 408 and the connector portion ring 414. It will be appreciated that the ring 410 and connector portion tip 416, as well as the tip 408 and connector portion ring 414, may be electrically connected by any suitable means and the present disclosure is in no way limited to the illustrative schematic. Therefore, for an electrode in electrical communication with the receptacle 404, the electrode ring acts as an cathode and the electrode tip acts as an anode. Advantageously, the adapter 400 may be used where an electrode having a conventional polarity configuration (i.e., where the tip acts as the cathode and the ring acts as the anode) initially causes phrenic nerve stimulation. By reversing the polarity of such an electrode, phrenic nerve stimulation may be reduced or eliminated.

While the present disclosure has been described in connection with what is considered the most practical and preferred embodiment, it is understood that this disclosure is not limited to the disclosed embodiments, but is intended to cover various arrangements included within the spirit and scope of the broadest interpretation so as to encompass all such modifications and equivalent arrangements. For example, and without limitation, while the illustrative receptacles disclosed herein are generally shown as bipolar, it will be appreciated that the receptacles may be configured to receive any suitable multipolar electrode, and include various permutations of parallel connections between receptacles, and remain within the scope of the present disclosure. Further, it will be appreciated that parallel connections among the receptacles may be arranged among any suitable number of receptacles. Moreover, and without limitation, while the illustrative connector portions disclosed herein are generally shown as bipolar, it will be appreciated that the connector portions may be configured as multipolar and remain within the scope of the present disclosure.

What is claimed is:

1. An adapter for coupling two or more electrodes to an electrostimulation device, the adapter comprising:
a yoke portion;
a connector portion extending distally from the yoke portion, the connector portion including a lead connector having a ring and a tip, wherein the connector portion is configured for reception by an electrostimulation device;
a first receptacle configured to accept an electrode and connected to the yoke portion, the first receptacle including a ring and a tip, wherein the ring is in electrical communication with the connector portion ring, and wherein the tip is in electrical communication with the connector portion tip; and
a second receptacle configured to accept an electrode and connected to the yoke portion, the second receptacle having a ring and a tip, wherein one of the tip and the ring is in electrical communication with the connector portion tip, and wherein the other of the tip and the ring is electrically isolated.

2. The adapter of claim 1 wherein the second receptacle ring is in electrical communication with the connector portion tip.

3. The adapter of claim 2 wherein the second receptacle tip is electrically isolated from the connector portion tip and the connector portion ring.

4. The adapter of claim 2 wherein the second receptacle ring is electrically parallel with the first receptacle tip.

5. The adapter of claim 1 wherein the second receptacle tip is in electrical communication with the connector portion tip.

6. The adapter of claim 5 wherein the second receptacle ring is electrically isolated from the connector portion ring and connector portion tip.

7. The adapter of claim 5 wherein the second receptacle tip is electrically parallel with the first receptacle tip.

8. The adapter of claim 1 further comprising an adjustable resistor in electrical communication with the connector portion and the first receptacle, the adjustable resistor operable to selectively adjust the current density to a lead in electrical communication with the first receptacle.

9. The adapter of claim 1 further comprising an adjustable resistor in electrical communication with the connector portion and the second receptacle, the adjustable resistor operable to selectively adjust the current density to a lead in electrical communication with the second receptacle.

10. The adapter of claim 1 further comprising a first electrode for implantation within a patient, the first electrode in electrical communication with the first receptacle; and a second electrode for implantation within a left ventricle of a patient, the second electrode in electrical communication with the second receptacle.

11. A method of cardiac electrostimulation of a patient with a cardiac electrostimulation device, the method comprising:
implanting a first electrode in the patient; and
implanting a second electrode in the left ventricle of the patient, wherein the first electrode and second electrode are electrically connected in parallel, and wherein the first electrode and second electrode share a common anode.

12. The method of claim 11 wherein the second electrode comprises a tip, and wherein the first electrode comprises a ring and a tip, wherein the second electrode tip and first electrode tip are electrically connected in parallel.

13. The method of claim 11 wherein the second electrode comprises a ring, and wherein the first electrode comprises a ring and a tip, wherein the second electrode ring and first electrode tip are electrically connected in parallel.

14. The method of claim 11 wherein the first electrode is implanted in one of the patient's right ventricle, left ventricle, superior vena cava, a cardiac vein, coronary sinus, or the great cardiac vein.

15. The method of claim 11 further comprising implanting an electrode in the right ventricle of the patient.

16. The method of claim 15 further comprising implanting an electrode in the right atrium of the patient.

17. A cardiac electrostimulation device comprising:
a cardiac electrostimulator;
a first receptacle for receiving a first electrode, the first receptacle in electrical communication with the electrostimulator, the first receptacle including a ring portion and a tip portion;
a second receptacle for receiving a second electrode, the second receptacle in electrical communication with the electrostimulator, the second receptacle including a ring portion and a tip portion;
a third receptacle for receiving a third electrode for implantation within the left ventricle of a patient, the third receptacle in electrical communication with the electrostimulator, the third receptacle including a tip portion; and
a fourth receptacle for receiving a fourth electrode, the fourth receptacle in electrical communication with the electrostimulator, the fourth receptacle including a ring portion and a tip portion.

18. The device of claim 17 wherein the third receptacle tip portion is electrically connected in parallel with the fourth receptacle tip portion, and wherein the fourth receptacle ring portion serves as a common anode for the third and fourth receptacles.

19. A cardiac electrostimulation device comprising:
a cardiac electrostimulator;
a first receptacle for receiving a first electrode, the first receptacle in electrical communication with the electrostimulator, the first receptacle including a ring portion and a tip portion;
a second receptacle for receiving a second electrode, the second receptacle in electrical communication with the electrostimulator, the second receptacle including a ring portion and a tip portion;
a third receptacle for receiving a third electrode for implantation within the left ventricle of a patient, the third receptacle in electrical communication with the electrostimulator, the third receptacle including a ring portion; and
a fourth receptacle for receiving a fourth electrode, the fourth receptacle in electrical communication with the electrostimulator, the fourth receptacle including a ring portion and a tip portion.

20. The device of claim 19 wherein the third receptacle ring portion is electrically connected in parallel with the fourth receptacle tip portion, and wherein the fourth receptacle ring portion serves as a common anode for the third and fourth receptacles.

21. An adapter for coupling an electrode to an electrostimulation device, the adapter comprising:
a connector portion having a unipolar lead with a tip, wherein the connector portion is configured for reception by an electrical stimulation device; and
a receptacle configured to accept an electrode and connected to the connector portion, the receptacle having a ring and a tip, wherein the ring is in electrical communication with the connector portion tip, and wherein the receptacle tip is in electrical isolation.

22. The adapter of claim 21 further comprising an electrode for implantation within a left ventricle of a patient, the electrode in electrical communication with the receptacle.

23. An adapter for coupling an electrode to an electrostimulation device, the adapter comprising:
a connector portion including a lead connector having a ring and a tip, wherein the connector portion is configured for reception by an electrostimulation device, wherein the ring is configured for electrical communication with an anode of the electrostimulation device, and wherein the tip is configured for electrical communication with a cathode of the electrostimulation device; and
a receptacle configured to accept an electrode and in electrical communication with the connector portion, the receptacle including a ring and a tip, wherein the ring is in electrical communication with the connector portion tip, and wherein the tip is in electrical communication with the connector portion ring.
